(19) United States
(12) Patent Application Publication
(10) Pub. No.: US 2026/0263082 A1
(43) Pub. Date: Sep. 10, 2026

(54) MULTIPLE CLIP LOADING SYSTEM

(71) Applicant: CILAG GMBH INTERNATIONAL, Zug (CH)

(72) Inventors: Daniel MUMAW, Liberty Township, OH (US); Kyle R. FLAKNE, Cincinnati, OH (US); Jeffrey P. WILEY, Milford, OH (US)

(73) Assignee: CILAG GMBH INTERNATIONAL, Zug (CH)

(21) Appl. No.: 19/071,526

(22) Filed: Mar. 5, 2025

(51) Int. Cl. A61B 17/128; A61B 17/28

(52) U.S. Cl. CPC A61B 17/1285; A61B 17/282

(57) ABSTRACT

A surgical system is disclosed that includes a surgical instrument insertable through a first trocar into a body cavity of a patient and a clip magazine insertable through a second trocar into the body cavity. The surgical instrument includes an end effector having opposing first and second jaws. The clip magazine includes a surgical clip. The end effector is interactable with the clip magazine within the body cavity to load the surgical clip between the opposing first and second jaws.

### BACKGROUND

The present disclosure relates to surgical systems and, more particularly, to surgical assemblies designed to dispense surgical clips within a patient cavity to be retrieved by surgical clip appliers.

Endoscopic surgical clip appliers may be used for a number of surgical procedures. In endoscopic or laparoscopic surgical procedures, access to the surgical site may be achieved through a trocar inserted through a small entrance incision in the skin. The trocar port allows the surgeon to insert a number of different surgical instruments therethrough and to perform surgical procedures within the patient in a minimally invasive manner.

Some surgical clip appliers are pre-loaded with a surgical clip prior to introducing the clip applier to the surgical site of a patient. After the surgical clip has been placed, the clip applier is typically removed from the patient and a new clip is loaded between the clip applier jaws and the clip applier is reintroduced into the patient. This removal and reintroduction of the clip applier is laborious and time consuming.

Accordingly, systems and methods for loading clips into the jaws of a clip applier while the clip applier remains within a patient are desired.

### BRIEF DESCRIPTION OF THE DRAWINGS

The following figures are included to illustrate certain aspects of the present disclosure, and should not be viewed as exclusive embodiments. The subject matter disclosed is capable of considerable modifications, alterations, combinations, and equivalents in form and function, without departing from the scope of this disclosure.

FIG. 1 is a block diagram of an example robotic surgical system that may incorporate some or all of the principles of the present disclosure.

FIG. 2 is an isometric top view of an example surgical tool that may incorporate some or all of the principles of the present disclosure.

FIG. 3 is an isometric bottom view of the surgical tool of FIG. 2.

FIG. 4 is an exposed isometric view of the surgical tool of FIG. 2.

FIG. 5 is an enlarged, isometric view of one example of the jaws of FIG. 4.

FIG. 6 is an enlarged isometric view of an example cam.

FIGS. 7A and 7B illustrate example operation of the cam and the jaws.

FIG. 8 is a block diagram of an example surgical system including the surgical tool of FIG. 2 and a clip magazine inserted into the body cavity of a patient via respective trocars, in accordance with at least one aspect of the present disclosure.

FIG. 9 is an isometric view of an example clip magazine for use with the surgical system of FIG. 8, in accordance with at least one aspect of the present disclosure.

FIG. 10A is a side cross-sectional view of another example clip magazine for use with the surgical system of FIG. 8, according to one or more additional embodiments.

FIG. 10B is an isometric view of portions of the clip magazine of FIG. 10A, in accordance with at least one aspect of the present disclosure.

FIG. 10C is an isometric view of clip holders of the clip magazine of FIG. 10A in a stowed position, in accordance with at least one aspect of the present disclosure.

FIG. 10D is an isometric view of the clip holders of FIG. 10C in a deployed position, in accordance with at least one aspect of the present disclosure.

FIG. 10E is a size view of the clip holders of FIG. 10C in the stowed position and an actuator of the clip magazine in a first, retracted position, in accordance with at least one aspect of the present disclosure.

FIG. 10F is a size view of the clip holders of FIG. 10E in the deployed position and the actuator in a second, extended position, in accordance with at least one aspect of the present disclosure.

FIG. 11 is a side cross-sectional view of another example clip magazine for use with the surgical system of FIG. 8, in accordance with at least one aspect of the present disclosure.

### DETAILED DESCRIPTION

The present disclosure relates to surgical systems and, more particularly, to assemblies designed to dispense surgical clips within a patient cavity to be retrieved by surgical clip appliers.

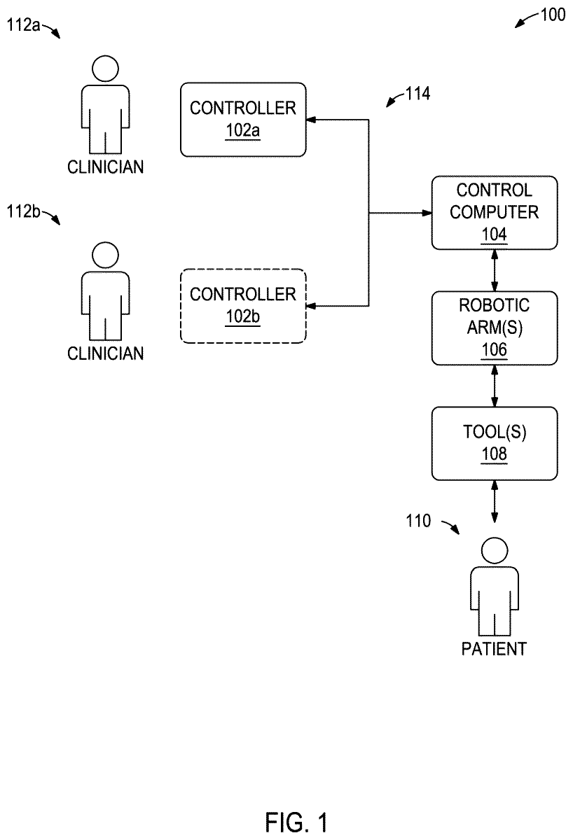

FIG. 1 is a block diagram of an example robotic surgical system 100 that may incorporate some or all of the principles of the present disclosure. As illustrated, the system 100 can include at least one master controller 102a and at least one arm cart 104. The arm cart 104 may be mechanically and/or electrically coupled to a robotic manipulator and, more particularly, to one or more robotic arms 106 or “tool drivers”. Each robotic arm 106 may include and otherwise provide a location for mounting one or more surgical tools or instruments 108 for performing various surgical tasks on a patient 110. Operation of the robotic arms 106 and instruments 108 may be directed by a clinician 112a (e.g., a surgeon) from the master controller 102a.

In some embodiments, a second master controller 102b (shown in dashed lines) operated by a second clinician 112b may also direct operation of the robotic arms 106 and instruments 108 in conjunction with the first clinician 112a. In such embodiments, for example, each clinician 102a,b may control different robotic arms 106 or, in some cases, complete control of the robotic arms 106 may be passed between the clinicians 102a,b. In some embodiments, additional arm carts (not shown) having additional robotic arms (not shown) may be utilized during surgery on a patient 110, and these additional robotic arms may be controlled by one or more of the master controllers 102a,b.

The arm cart 104 and the master controllers 102a,b may be in communication with one another via a communications link 114, which may be any type of wired or wireless telecommunications means configured to carry a variety of communication signals (e.g., electrical, optical, infrared, etc.) according to any communications protocol.

The master controllers 102a,b generally include one or more physical controllers that can be grasped by the clinicians 112a,b and manipulated in space while the surgeon views the procedure via a stereo display. The physical controllers generally comprise manual input devices movable in multiple degrees of freedom, and which often include an actuatable handle for actuating the surgical instrument(s) 108, for example, for opening and closing opposing jaws, applying an electrical potential (current) to an electrode, or the like. The master controllers 102a,b can also include an optional feedback meter viewable by the clinicians 112a,b via a display to provide a visual indication of various surgical instrument metrics, such as the amount of force being applied to the surgical instrument (i.e., a cutting instrument or dynamic clamping member).

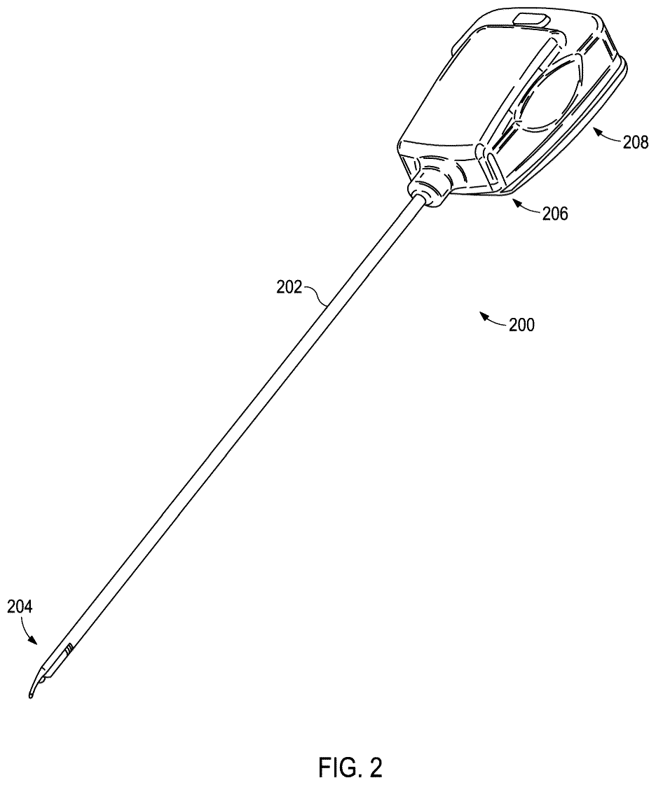

FIG. 2 is an isometric top view of an example surgical tool or “surgical instrument” 200 that may incorporate some or all of the principles of the present disclosure. The surgical tool 200 may be the same as or similar to the surgical instrument(s) 108 of FIG. 1 and, therefore, may be used in conjunction with the robotic surgical system 100 of FIG. 1. Accordingly, the surgical tool 200 may be designed to be releasably coupled to a robotic arm 106 (FIG. 1) of a robotic manipulator of the robotic surgical system 100. Full detail and operational description of the surgical tool 200 is provided in co-owned U.S. Pat. No. 10,039,548, entitled “Clip Applier Adapted for Use with a Surgical Robot”.

While the surgical tool 200 is described herein with reference to a robotic surgical system, it is noted that the principles of the present disclosure are equally applicable to non-robotic surgical tools or, more specifically, manually operated surgical tools. Accordingly, the discussion provided herein relating to robotic surgical systems merely encompasses one example application of the presently disclosed inventive concepts.

As illustrated, the surgical tool 200 can include an elongate shaft 202, an end effector 204 coupled to the distal end of the shaft 202, and a drive housing 206 provided at the proximal end of the shaft 202. The terms “proximal” and “distal” are defined herein relative to a robotic surgical system having an interface configured to mechanically and electrically couple the surgical tool 200 (e.g., the drive housing 206) to a robotic manipulator. The term “proximal” refers to the position of an element closer to the robotic manipulator and the term “distal” refers to the position of an element closer to the end effector 204 and thus further away from the robotic manipulator. Moreover, the use of directional terms such as above, below, upper, lower, upward, downward, left, right, and the like are used in relation to the illustrative embodiments as they are depicted in the figures, the upward or upper direction being toward the top of the corresponding figure and the downward or lower direction being toward the bottom of the corresponding figure.

In applications where the surgical tool 200 is used in conjunction with a robotic surgical system (e.g., system 100 of FIG. 1), the drive housing 206 can include a tool mounting portion 208 designed with features that releasably couple the surgical tool 200 to a robotic arm (e.g., the robotic arms 106 or “tool drivers” of FIG. 1) of a robotic manipulator. The tool mounting portion 208 may releasably attach (couple) the drive housing 206 to a tool driver in a variety of ways, such as by clamping thereto, clipping thereto, or slidably mating therewith. In some embodiments, the tool mounting portion 208 may include an array of electrical connecting pins, which may be coupled to an electrical connection on the mounting surface of the tool driver. While the tool mounting portion 208 is described herein with reference to mechanical, electrical, and magnetic coupling elements, it should be understood that a wide variety of telemetry modalities might be used, including infrared, inductive coupling, or the like.

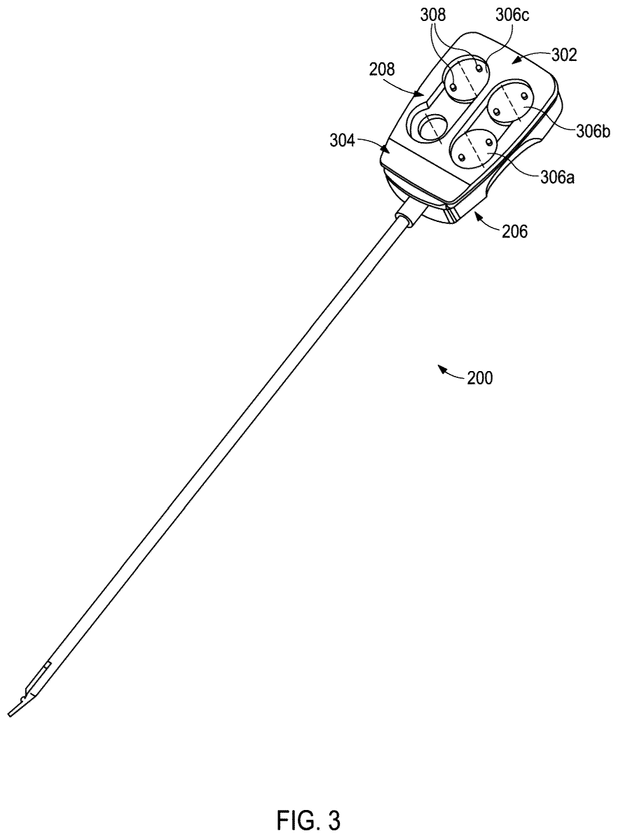

FIG. 3 is an isometric bottom view of the surgical tool 200. As illustrated, the surgical tool 200 further includes an interface 302 that mechanically and electrically couples the tool mounting portion 208 to a robotic manipulator. In various embodiments, the tool mounting portion 208 includes a tool mounting plate 304 that operably supports a plurality of drive inputs, shown as a first drive input 306a, a second drive input 306b, and a third drive input 306c. While only three drive inputs 306a-c are shown in FIG. 3, more or less than three may be employed, without departing from the scope of the disclosure.

In the illustrated embodiment, each drive input 306a-c comprises a rotatable disc configured to align with and couple to a corresponding input actuator (not shown) of a given tool driver. Moreover, each drive input 306a-c provides or defines one or more surface features 308 configured to align with mating surface features provided on the corresponding input actuator. The surface features 308 can include, for example, various protrusions and/or indentations that facilitate a mating engagement.

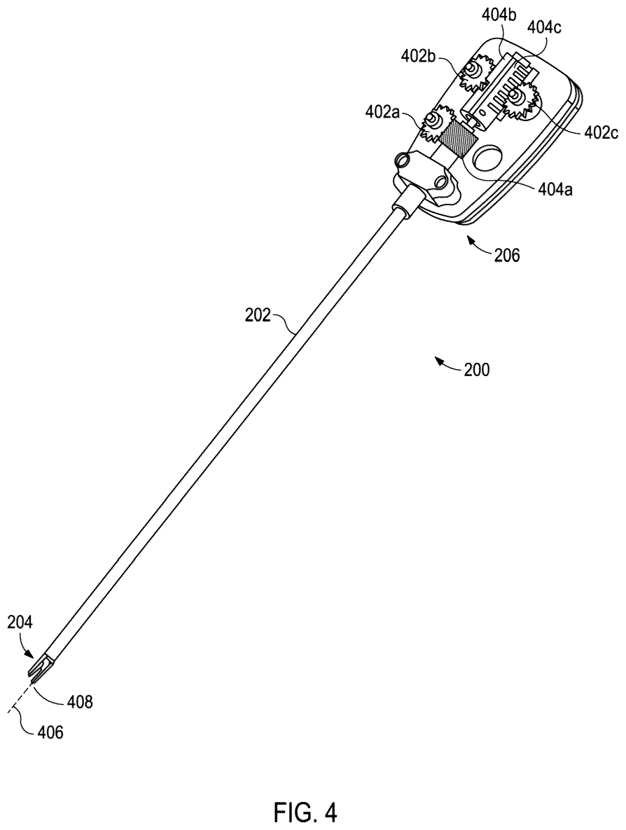

FIG. 4 is an exposed isometric view of the surgical tool 200. The shroud that covers the drive housing 206 has been removed to reveal the internal component parts. As illustrated, the surgical tool 200 may include a first drive gear 402a, a second drive gear 402b, and a third drive gear 402c. The first drive gear 402a may be operatively coupled to (or extend from) the first drive input 306a (FIG. 3) such that actuation of the first drive input 306a correspondingly rotates the first drive gear 402a. Similarly, the second and third drive gears 402b,c may be operatively coupled to (or extend from) the second and third drive inputs 306b,c (FIG. 3), respectively, such that actuation of the second and third drive inputs 306b,c correspondingly rotates the second and third drive gears 402b,c, respectively.

The first drive gear 402a may be configured to intermesh with a first driven gear 404a, which is operatively coupled to the shaft 202. In the illustrated embodiment, the driven gear 404a comprises a helical gear. In operation, rotation of the first drive gear 402a correspondingly rotates the first driven gear 404a about a longitudinal axis 406 of the shaft 202. The first driven gear 404a is coupled to the shaft 202 such that rotation of the first drive gear 402a controls rotation of the shaft 202 in clockwise and counter-clockwise directions based on the rotational direction of the first drive gear 402a.

The second drive gear 402b may be configured to intermesh with a second driven gear 404b (partially visible in FIG. 4), and the third drive gear 402c may be configured to intermesh with a third driven gear 404c. In the illustrated embodiment, the second and third drive and driven gears 402b,c and 404b,c comprise corresponding rack and pinion interfaces, where the driven gears 404b,c comprise the rack and the drive gears 402b,c comprise the corresponding pinion. Independent rotation of the second and third drive gears 402b,c will cause the second and third driven gears 404b,c, respectively, to translate linearly relative to (independent of) one another.

In the illustrated embodiment, the surgical tool 200 comprises a surgical clip applier and the end effector 204 has opposing first and second jaws 408a, 408b. Actuation of the second drive gear 402b causes the jaws 408 to close or collapse to crimp a surgical clip residing therebetween. Once the surgical clip is successfully deployed (crimped), rotation of the second drive gear 402b is reversed in the opposite angular direction to move the second driven gear 404b in a proximal direction, which permits the jaws 408 to open once again in preparation for receiving another surgical clip. The process may be repeated several times to feed and crimp a predetermined number of clips residing in the shaft 202.

It should be noted that the processes of delivering a surgical clip into the jaws 408 and collapsing the jaws 408 to crimp the surgical clip are not limited to the actuation mechanisms and structures described herein. In alternative embodiments, for example, the second and third driven gears 404b,c may instead comprise capstan pulleys configured to route and translate drive cables within the shaft 202. In such embodiments, the drive cables may be operatively coupled to one or more lead screws or other types of rotating members positioned within the shaft 202 near the distal end and capable of advancing a feedbar that delivers a surgical clip into the jaws 408 and advancing a cam to collapse the jaws 408 and crimp the surgical clip.

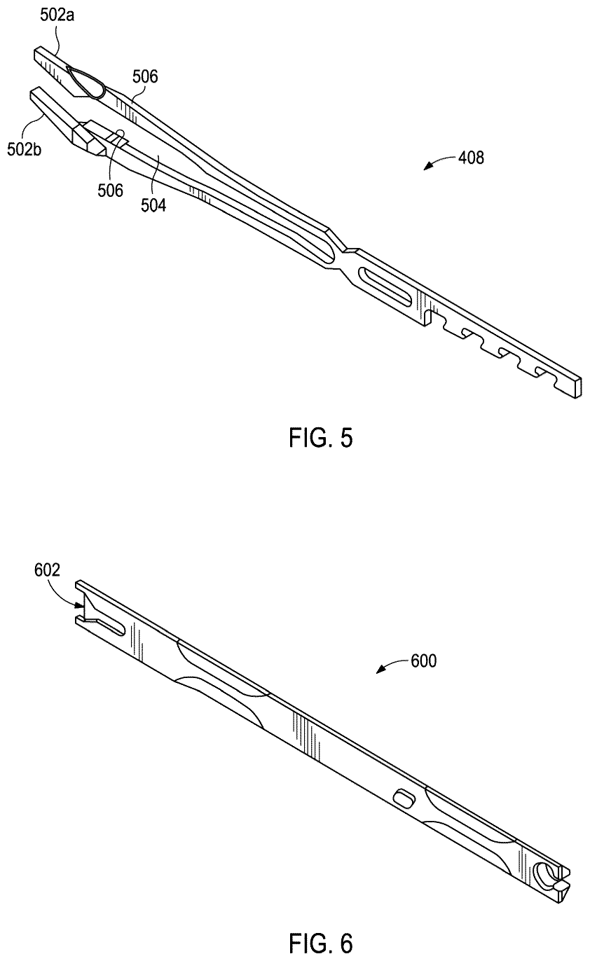

FIG. 5 is an enlarged, isometric view of one example of the jaws 408 of FIG. 4. In the illustrated embodiment, the jaws 408 comprise a one-piece architecture having opposed first and second jaws 502a and 502b with a gap 504 defined therebetween. The first and second jaws 502a,b are movable relative to one another and adapted to receive a surgical clip (not shown) therebetween. The jaws 502a,b may be naturally biased to an open position, and a force is required to move the jaws 502a,b toward one another (i.e., collapse the jaws 408). In some applications, each jaw 502a,b can include a groove formed on opposed inner surfaces thereof for receiving the legs of a surgical clip in alignment with the jaws 502a,b. Each jaw 502a,b can also include a cam track 506 formed thereon. The cam tracks 506 are essentially ramped features formed on a superior (upper) surface of each jaw 502a,b. In the illustrated embodiment, a cam 600 (FIG. 6) may be configured to engage the cam tracks 506 and thereby urge (force) the jaws 502a,b to collapse toward one another.

FIG. 6 is an enlarged isometric view of one example of the cam 600. The cam 600 may be configured for slidably mating with and engaging the jaws 502a,b (FIG. 6). More specifically, the cam 600 may provide or otherwise define a camming channel 602 adapted to engage and actuate the jaws 408 (FIGS. 4 and 5). As illustrated, the camming channel 602 forms a tapering recess. During actuation, the camming channel 602 is configured to slidably receive the cam tracks 506 (FIG. 5) provided by the jaws 502a,b (FIG. 5). Distal movement of the cam 600 relative to the jaws 408 will urge the jaws 502a,b to collapse toward each other.

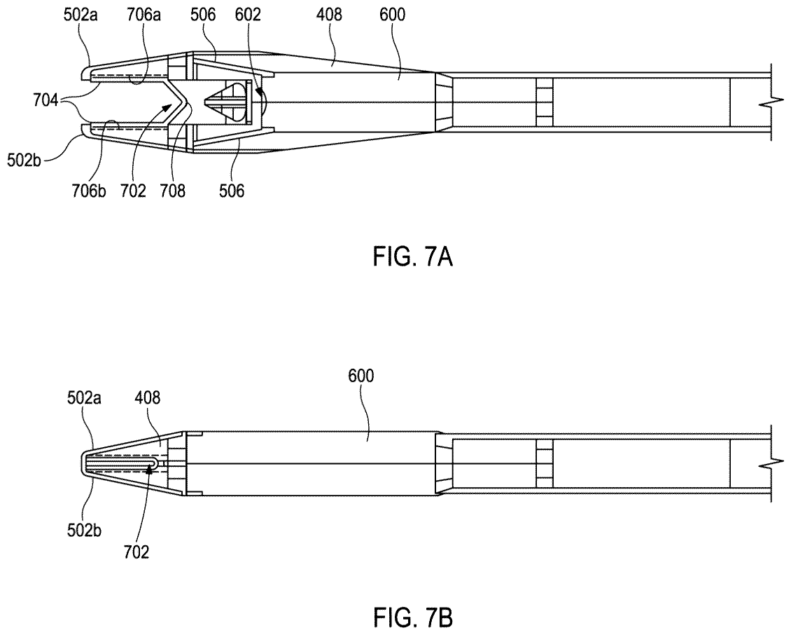

FIGS. 7A and 7B illustrate example operation of the cam 600 and the jaws 408. In FIG. 7A, a surgical clip 702 has been positioned between the jaws 408. As illustrated, first and second legs 704a,b of the surgical clip 702 are received within corresponding first and second grooves 706a,b defined in the opposed inner surfaces of the jaws 502a,b, and the crown 708 (alternately referred to as the “apex”) is positioned between the jaws 502a,b and points proximally.

To crimp the surgical clip 702, the cam 600 is advanced distally (i.e., to the left in FIGS. 7A-7B) relative to the jaws 408. In FIG. 7A, the cam 600 is shown in a proximal position, where the jaws 502a,b are spaced apart from one another in an “open” configuration. As the cam 600 is advanced distally over the jaws 502a,b, the camming channel 602 receives and slidingly engages the angled surfaces of the cam tracks 506, which simultaneously urges the jaws 502a,b to collapse toward one another (to a “closed” configuration) and crimp the surgical clip 702. FIG. 7B shows the crimped surgical clip 702.

During a surgical procedure, opposing jaws of a surgical tool (e.g. opposing jaws 408 of surgical tool 200) may place individual clips (e.g. surgical clips 702) onto the tissue of a patient. After a clip has been placed, the jaws of the surgical instrument are typically removed from the patient and a new clip is placed therein before the jaws are reintroduced into the patient. This removal and reintroduction of the jaws is laborious and time consuming. Accordingly, systems and methods for loading clips into the jaws of a surgical tool are desired.

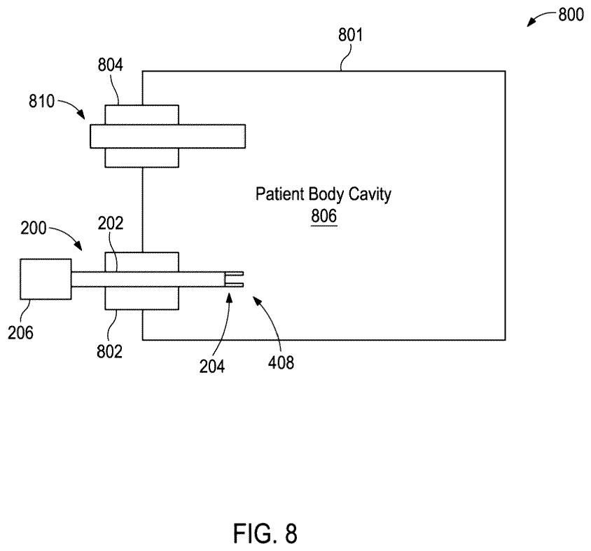

FIG. 8 is a schematic block diagram of an example surgical system 800, in accordance with at least one aspect of the present disclosure. As illustrated, the surgical system 800 may include first and second trocars 802, 804 each inserted through the skin of a patient 801 and extending to a body cavity 806 defined within the patient 801. While only two trocars 802, 804 are shown and described herein, the surgical system 800 may include more than two trocars (e.g., three, four, or five trocars) such that additional surgical instruments may be inserted into the body cavity 806.

The surgical tool 200 may be advanced into the body cavity 806 through the first trocar 802, thereby allowing the surgical tool 200 to apply surgical clips 702 (FIG. 7) to tissue located in the body cavity 806. According to embodiments of the present disclosure, a clip cartridge or “magazine” 810 may be inserted through the second trocar 804. The clip magazine 810 may include and otherwise store therein one or more surgical clips 702 that may be accessed by the opposing jaws 408 of the surgical tool 200 while within the body cavity 806. More specifically, the first and second jaws 502a,b (FIG. 5) of the surgical tool 200 may access and interact with the clip magazine 810 within the body cavity 806 to load a surgical clip 702 (FIG. 7) therebetween. Accordingly, the clip magazine 810 enables a user to reload surgical clips 702 into the opposing jaws 408 of the surgical tool 200 within the body cavity 806 without withdrawing the end effector 204 from the first trocar 802.

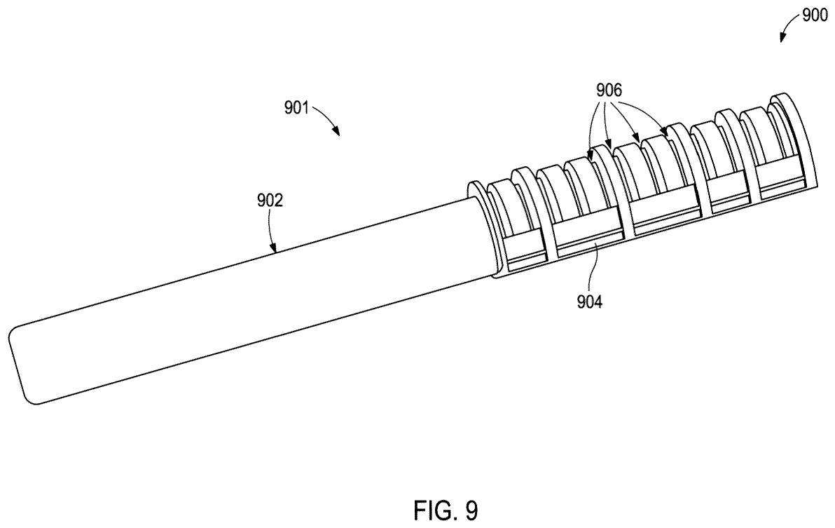

FIG. 9 is an isometric view of an example clip magazine 900 for use with the surgical system 800 of FIG. 8, in accordance with at least one aspect of the present disclosure. The clip magazine 900 may be the same as or similar to the clip magazine 800 of FIG. 8, and therefore may be able to be introduced into the body cavity 806 (FIG. 8) through a trocar (e.g., the second trocar 802). As illustrated, and with reference to both FIGS. 8 and 9, the clip magazine 900 may comprise a body 901 that includes a first body portion 902 and a second body portion 904 extending from the first body portion 902. The first body 901 may be advanced through the second trocar 804 to introduce (position) the second body portion 904 within the body cavity 806. In some embodiments, and once the body 901 is properly placed within the body cavity 806, the first body portion 902 may be secured to the second trocar 804, such as via a latch, magnets, fasteners, etc., to prevent the clip magazine 900 from moving relative to the second trocar 804. Alternatively, the clip magazine 900 may be movable relative to the second trocar 804 to allow a user to navigate (move) the clip magazine 900 toward or away from the surgical instrument 200 within the body cavity 806.

The second body portion 904 may define a plurality of slots 906 along the length thereof. Each slot 906 may be sized to receive and store therein a discrete surgical clip 702 (FIG. 7). In use, the position of the end effector 204 may be manipulated within the body cavity 806 to locate and interact with the clip magazine 900 to load (position) a surgical clip 702 between the opposing jaws 408. More specifically, within the body cavity 806, a user may navigate one or both of the end effector 204 and the clip magazine 900 toward one another, and the tips of the opposing jaws 408 may be inserted into a desired slot 906 in the second body portion 904. Advancing the jaws 408 into the slot 906 may cause a surgical clip 702 to be received between the opposing jaws 408. The surgical tool 200 may then be navigated (moved) away from clip magazine 900 to apply the “loaded” clip 702 onto patient tissue within the body cavity 806. The aforementioned process may be repeated to reload additional clips 702 into the opposing jaws 408 within the clip magazine 900.

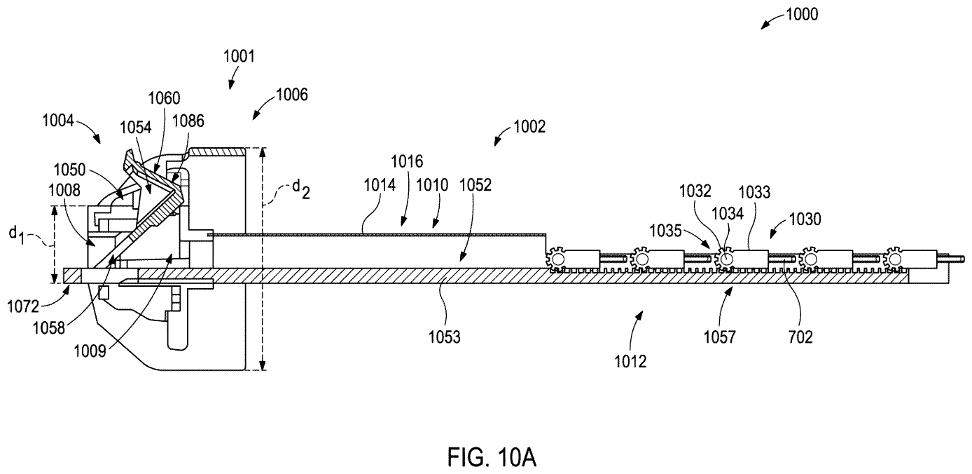

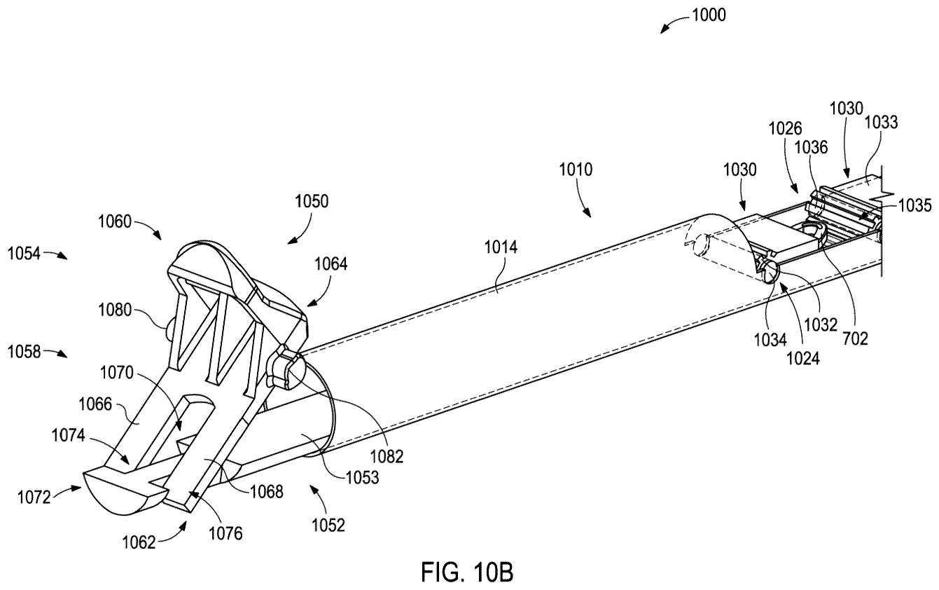

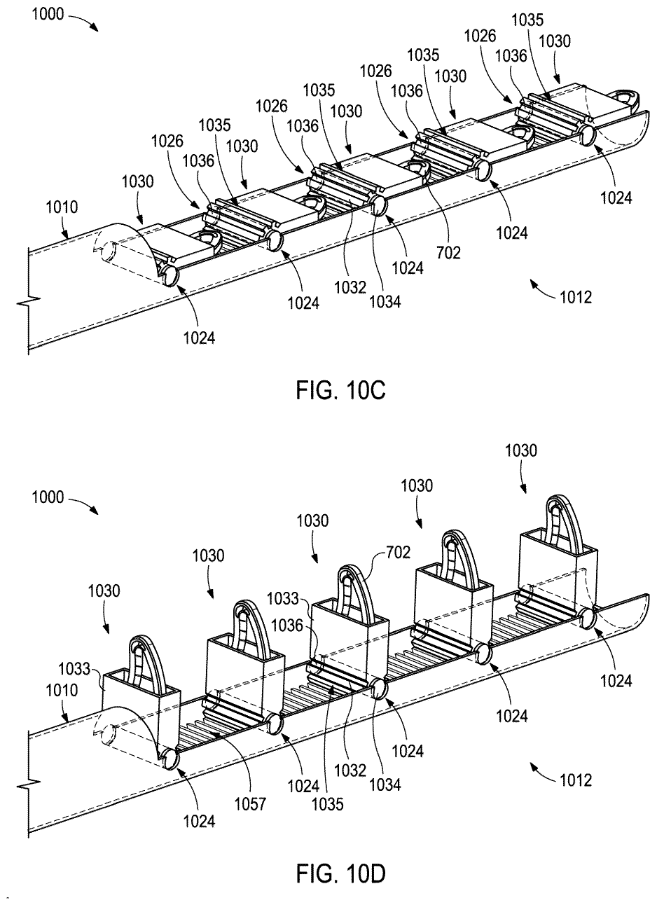

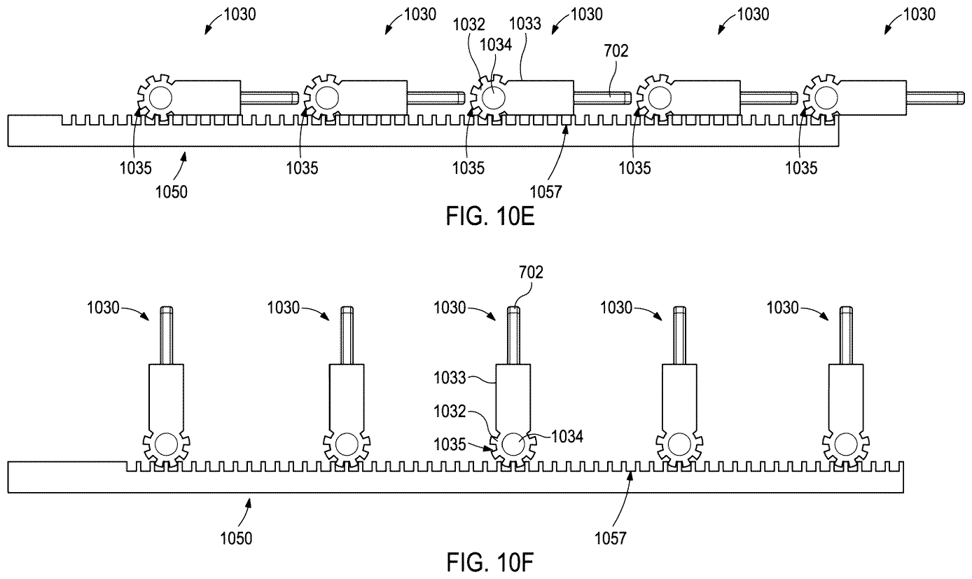

FIG. 10A is a cross-sectional side view of another example clip magazine 1000 that may be used with the surgical system 800 of FIG. 8, according to one or more additional embodiments. The clip magazine 1000 may be the same as or similar to the clip magazine 800 of FIG. 8, and therefore may be able to be introduced into the body cavity 806 (FIG. 8) through a trocar (e.g., the second trocar 802).

As illustrated, the clip magazine 1000 may include a head or “cap” 1001 and a shaft 1002 extending from the head 1001. The head 1001 may include a “dome-like” shape that defines a first diameter d₁ at a first or “proximal” end 1004 thereof and a second diameter d₂ different (e.g., greater) than the first diameter d₁ at a second or “distal” end 1006 opposite the first end 1004. The second diameter d₂ may be the same as or greater than a diameter of the second trocar 804 (FIG. 8), such that when the shaft 1002 is introduced into the body cavity 806 (FIG. 8) via the second trocar 804, the second end 1006 of the head 1001 may abut or receive the second trocar 804 to thereby prevent the head 1001 from passing through the second trocar 804 and into the body cavity 806. In some applications, the head 1001 may be secured to the second trocar 804, such as via a latch, magnets, fasteners, etc., to prevent the head 1001 from moving relative to the second trocar 804. The head 1001 may further define an aperture 1008 at the first end 1006 and an interior cavity 1009.

The shaft 1002 may include a first shaft portion 1010 and a second shaft portion 1012 extending from the first shaft portion 1010. The first shaft portion 1010 may comprise a hollow shaft 1014 operatively coupled to the head 1001 and defining an interior channel 1016 in communication with the interior cavity 1009. As best seen in FIGS. 10C and 10D, the second shaft portion 1012 may comprise a U-shaped extension extending from the first shaft portion 1010, thereby exposing the interior of the second shaft portion 1012.

Referring to FIGS. 10C and 10D, the second shaft portion 1012 may define a plurality of longitudinally spaced first apertures 1024 and a plurality of longitudinally spaced second apertures 1026, where each first aperture 1024 laterally aligns with a corresponding one of the second apertures 1024. The clip magazine 1000 may further include a plurality of clip holders 1030 rotatably mounted to the shaft 1002 at the first and second apertures 1024, 1026. More specifically, each clip holder 1030 may include a shaft 1032 and a receptacle 1033 extending from the shaft 1032. While five clip holders 1030 are shown, the clip magazine 1000 may have less (e.g. two, three or four) or more (e.g. six, eight, or ten) than five clip holders 1030.

Each shaft 1032 may define, or otherwise provide, a first pin 1034 extending from a first side thereof and a second pin 1036 extending from a second side thereof. The first pin 1034 may be received in the first aperture 1024 and the second pins 103 may be received in the second aperture 1026, thereby rotatably coupling the clip holders 1030 to the second shaft portion 1012 and allowing the clip holders 1030 to rotate between a first or “stowed” position, as shown in FIGS. 10C and 10E, and a second or “deployed” position, as shown in FIGS. 10D and 10F. In the stowed position, the clip holders 1030 may be substantially aligned with the longitudinal axis of the second shaft portion 1012 such that no angle is defined therebetween. In contrast, in the deployed position, the clip holders 1030 may be angled (rotated) away from the second shaft portion 1012 such that a non-zero angle is defined therebetween. In at least one embodiment, for example, the clip holders 1030 may be perpendicular (rotated 90°) to the second shaft portion 1012 when actuated to the deployed state. However, other embodiments are envisioned where the clip holders 1030 are rotated to a non-zero angle less than 90° from the second shaft portion 1012 in the deployed state (e.g., 30°, 45°, 60°, 75°).

Each shaft 1032 may define, or otherwise provide, a plurality of teeth 1035 around a portion of the perimeter thereof. Each receptacle 1033 may define a recess or pocket sized to receive a surgical clip 702 therein.

Referring again to FIG. 10A, the clip magazine 1000 may further include an actuation system 1050 for rotating the clip holders 1030 between the stowed and deployed positions. As illustrated, the actuation system 1050 may include an actuator 1052 and a lever 1054 operatively coupled to the actuator 1052 such that actuating (operating) the lever 1052 causes the actuator 1052 to move longitudinally. In the illustrated embodiment, the actuator 1052 comprises a rack 1053 that extends from the interior 1009 of the head 1001, through the interior channel 1016 of the hollow shaft 1014, and to the second shaft portion 1012. The rack 1053 may provide or otherwise defines a plurality of teeth 1057 operable to mesh with the teeth 1035 provided by the clip holders 1030.

In example operation, the actuator 1052 may be longitudinally translatable along the shaft 1002 between a first or “retracted” position, as shown in FIGS. 10A-10C and 10E, and a second or “extended” position, as shown in FIGS. 10D and 10F. The retracted position of the actuator 1052 may correspond to the stowed position of the clip holders 1030 and the extended position of the actuator 1052 may correspond to the deployed position of the clip holders 1030. In use, when the actuator 1052 transitions to the extended position, the teeth 1057 on the rack 1053 drive against the teeth 1035 of the clip holders 1030, and thereby cause the clip holders 1030 to rotate to their respective deployed positions. Conversely, when the actuator 1052 transitions to the retracted position, the teeth 1057 on the rack 1053 drive against the teeth 1035 of the clip holders 1030, and thereby cause the clip holders 1030 to rotate toward their respective stowed positions.

FIG. 10B is an isometric view of the clip magazine 1000 with the head 1001 of the clip magazine 1000 omitted to enable a better view of the internal components of the actuation system 1050, such as the lever 1054. As illustrated, the lever 1054 includes a body 1058 and a tab 1060 extending from the body 1058. The body 1058 may include opposing first and second ends 1062 and 1064, and the tab 1060 is provided at the second end 1064 and may extend transversely therefrom.

In some embodiments, the first end 1062 may include first and second legs 1066, 1068 spaced from each other and thereby defining a gap 1070. The actuator 1052, and more particularly the rack 1053, may include an end 1072 that defines first and second notches 1074, 1076 on a second side thereof opposite the first side. The first notch 1074 may define a first aperture (occluded from view) and the second notch 1076 may define a second aperture (occluded from view). The first and second legs 1066, 1068 may extend into the first and second notches 1074, 1076, respectively, and may be rotatably coupled to the rack 1053 at the notches 1074. In some embodiments, for example, the first and second legs 1066, 1068 may be pinned to the rack 1053 at the first and second notches 1074, 1076, respectively, thereby rotatably coupling the lever 1054 to the actuator 1052.

Third and fourth pins 1080 and 1082 may extend from opposing lateral sides of the body 1058 at or near the second end 1064. The third and fourth pins 1080, 1082 may be received in corresponding first and second apertures defined in the head 1001 (FIG. 10A), thereby rotatably coupling the lever 1054 to the head 1001.

Referring again to FIG. 10A, the head 1001 may define an aperture 1086 through which the tab 1060 of the lever 1054 may extend. The tab 1060 may be manually graspable and otherwise actuatable by a user to act on the lever 1054 and thereby transition the actuator 1052 (e.g., the rack 1053) between the retracted and extended positions, which correspondingly transitions the clip holders 1030 between the stowed and deployed positions. In use, a user may rotate (actuate) the lever 1054 to advance the actuator 1052 toward the extended position, thereby rotating the clip holders 1030 to their respective deployed positions. Conversely, the user may rotate (actuate) the lever 1054 to retract the actuator 1052 toward the retracted position, thereby rotating the clip holders 1030 to their respective stowed positions. In some embodiments, the clip magazine 1000 may include a torsion spring to bias the lever 1054 toward a first position and/or a linear spring to bias the actuator 1052 toward the retracted position.

The clip magazine 1000 may be introduced into the body cavity 806 (FIG. 8) to provide surgical clips 702 for the end effector 204 (FIG. 8). For instance, once arranged within the body cavity 806, a user may navigate one or both of the opposing jaws 408 (FIG. 8) and the clip magazine 1000 toward one another. The user may actuate the lever 1054 to transition the clip holders 1030 to their respective deployed positions, thereby allowing the jaws 408 to be inserted into the receptacle 1033 of the clip holder 1030 to receive a corresponding one of the stored surgical clips 702. The surgical tool 200 may then be navigated (moved) away from clip magazine 1000 to apply the “loaded” clip 702 onto patient tissue within the body cavity 806. The aforementioned process may be repeated to reload additional surgical clips 702 into the opposing jaws 408 within the clip magazine 1000.

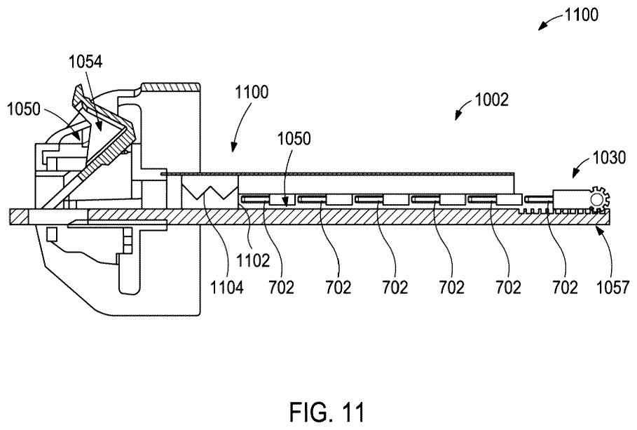

FIG. 11 is a cross-sectional side view of another example clip magazine 1100 that can be used with the surgical system 800 of FIG. 8, according to one or more additional embodiments. The clip magazine 1000 may be the same as or similar to the clip magazine 800 of FIG. 8, and therefore may be able to be introduced into the body cavity 806 (FIG. 8) through a trocar (e.g., the second trocar 802). Moreover, the clip magazine 1100 may be similar in some respects to the clip magazine 1000 of FIGS. 10A-10F, and therefore may be best understood with reference thereto, where like numerals will correspond to like components not described again in detail.

Unlike the clip magazine 1000 of FIGS. 10A-10F, which includes a plurality of clip holders 1030, the clip magazine 1100 includes a single clip holder 1030. The clip magazine 1100 further includes a feeder system 1100 extending through the shaft 1002 and configured to sequentially and systematically feed clips 702 into the holder 1030. The feeder system 1100 also includes a pusher plate 1102 and a spring 1104 operably coupled to and extending between the pusher plate 1102 and a portion of the head 1001. The spring 1104 may comprise a compression spring that is preloaded and operable to bias (push) the pusher plate 1102 away from head 1001 and toward the clip holder 1030.

A plurality of serially arranged surgical clips 702 may be arranged within the shaft 1002 and sequentially fed into the receptacle 1032 of the clip holder 1030, one-by-one, as the spring 1104 pushes against the pusher plate 1102. In example operation, the lever 1054 may be actuated to transition the clip holder 1030 to the stowed state, and otherwise placing the pocket defined by the receptacle in line with the serially arranged surgical clips 702. With the clip holder 1030 in the stowed state, the spring 1104 may bias the pusher plate 1102 to act (push) on the surgical clips 702, thereby causing the last surgical clip 702 to be loaded into the receptacle 1033 of the clip holder 1030. The lever 1054 may then be actuated to transition the clip holder 1030 back to the deployed state. With the clip holder 1030 in the deployed state, the surgical clip 702 may be received between the opposing jaws 408 (FIG. 8) of the end effector 204 (FIG. 8), as generally described above. Once the surgical clip 702 is properly received between the jaws 408 and removed from the receptacle 1033, the lever 1054 may be actuated again to transition the clip holder 1030 back to the stowed state to receive another (e.g., the penultimate) surgical clip 702 within the receptacle 1033.

Accordingly, the clip magazine 1100 provides a consistent position (i.e. at the single clip holder 1030) in which to load surgical clips 702 into the jaws 408 (FIG. 8) of the end effector 204 (FIG. 8). In some embodiments, the head 1001 or the shaft 1002 of the clip magazine 1100 may define an aperture that allows a user to feed additional surgical clips 702 into the shaft 1002.

Accordingly, the presently described clip magazines 900, 1000, 1100 enable a user to load a surgical clip into a surgical tool within a patient's body cavity, thereby no longer requiring the surgical tool to be removed from the patient each time a new clip is needed.

Embodiments disclosed herein include:

A. A surgical system comprising a surgical instrument insertable through a first trocar into a body cavity of a patient, the surgical instrument comprising an end effector having opposing first and second jaws, and a clip magazine insertable through a second trocar into the body cavity and including a surgical clip, wherein the end effector is interactable with the clip magazine within the body cavity to load the surgical clip between the opposing first and second jaws.

B. A surgical system comprising a surgical instrument insertable through a first trocar into a body cavity of a patient, the surgical instrument comprising and end effector having opposing first and second jaws, and a clip magazine insertable through a second trocar into the body cavity and including a shaft extendable through the second trocar and into the body cavity and clip holder rotatably mounted to the shaft and providing a receptacle sized to hold a surgical clip, wherein the end effector is interactable with the clip magazine within the body cavity to load the surgical clip between the opposing first and second jaws.

C. A method comprising advancing an end effector of a surgical instrument into a body cavity of a patient via a first trocar, the end effector including opposing first and second jaws, advancing a clip magazine into the body cavity of the patient via a second trocar, the clip magazine including a surgical clip, moving at least one of the end effector and the clip magazine within the body cavity to place then end effector adjacent the clip magazine, and loading the surgical clip between the first and second jaws within the body cavity.

Each of embodiments A-C may have one or more of the following additional elements in any combination: Element 1: wherein the clip magazine comprises a body defining a slot sized to receive the surgical clip, and wherein the opposing jaws are receivable within the slot to load the surgical clip between the opposing first and second jaws. Element 2: wherein the clip magazine comprises a shaft and a clip holder rotatably mounted to the shaft and defining a receptacle that receives the surgical clip, the clip holder being rotatable between a stowed position, where the clip holder is aligned with a longitudinal axis of the shaft, and a deployed position, where the clip holder is rotated out of alignment with the longitudinal axis. Element 3: wherein the clip magazine further comprises an actuator movable relative to the shaft between a retracted position, where the clip holder is rotated to the stowed position, and an extended position, where the clip holder is rotated toward the deployed position. Element 4: wherein the actuator comprises a rack comprising first teeth, the clip holder comprises second teeth arranged to intermesh with the second teeth, and the first teeth drive against the second teeth to rotate the clip holder between the stowed and deployed positions. Element 5: wherein the surgical clip is a first surgical clip and the surgical system further includes a feeder system operable to load a second surgical clip into the clip holder after the first surgical clip is removed from the clip holder. Element 6: wherein the second surgical clip is included in a plurality of serially arranged surgical clips, and the feeder system further includes a pusher plate engageable against the plurality of serially arranged surgical clips and a spring engageable against the pusher plate and operable to bias the pusher plate against the plurality of serially arranged surgical clips and thereby sequentially feed surgical clips of the plurality of serially arranged surgical clips into the receptacle of the clip holder. Element 7: wherein the clip holder is transitionable to the stowed state to receive a last one of the plurality of serially arranged surgical clips, and the deployed state, to allow the end effector to load the last one of the plurality of serially arranged surgical clips between the opposing first and second jaws.

Element 8: wherein the clip holder is rotatable between a stowed position where the clip holder is aligned with a longitudinal axis of the shaft, and a deployed position, where the clip holder is rotated out of alignment with the longitudinal axis. Element 9: wherein the clip magazine further comprises an actuator movable relative to the shaft between a retracted position, where the clip holder is rotated to the stowed position, and an extended position, where the clip holder is rotated toward the deployed position. Element 10: wherein the actuator comprises a rack comprising first teeth, the clip holder includes second teeth arranged to intermesh with the second teeth, and the first teeth drive against the second teeth to rotate the clip holder between the stowed and deployed positions. Element 11: wherein the surgical clip is a first surgical clip and wherein the surgical system further comprises a feeder system operable to load a second surgical clip into the clip holder after the first surgical clip is removed from the clip holder. Element 12: wherein the clip holder is a first clip holder and the surgical clip is a first surgical clip, and wherein the clip magazine further comprises a second clip holder rotatably coupled to the shaft and including a receptacle sized to hold a second surgical clip. Element 13: wherein the clip magazine further comprises an actuator movable relative to the shaft between a retracted position, where the first and second clip holders are each arranged in a stowed position, an extended position, where the first and second clip holders are rotated simultaneously toward a deployed position.

Element 14: wherein the clip magazine defines a slot sized to receive the surgical clip, and wherein loading the surgical clip between the first and second jaws comprises inserting the first and second jaws into the slot. Element 15: wherein the clip magazine comprises a clip holder that defines a receptable sized to receive the surgical clip, and wherein the method further comprises advancing an actuator through a shaft of the clip magazine, thereby rotating the clip holder toward a deployed state. Element 16: wherein loading the surgical clip between the first and second jaws comprises inserting the first and second jaws into the clip holder in the deployed state. Element 17: wherein the surgical clip comprises a first surgical clip and the clip magazine further includes a second surgical clip, the method further comprising applying the first surgical clip to tissue with the surgical instrument and loading the second surgical clip between the first and second jaws within the body cavity.

By way of non-limiting example, exemplary combinations applicable to A, B, and C include: Element 2 with Element 3; Element 2 with Elements 3 and 4; Element 2 with Elements 3-5; Element 2 with Elements 3-6; Element 2 with Elements 3-7; Element 8 with Element 9; Element 8 with Elements 9 and 10; Element 12 with Element 13; Element 15 with Element 16.

Therefore, the disclosed systems and methods are well adapted to attain the ends and advantages mentioned as well as those that are inherent therein. The particular embodiments disclosed above are illustrative only, as the teachings of the present disclosure may be modified and practiced in different but equivalent manners apparent to those skilled in the art having the benefit of the teachings herein. Furthermore, no limitations are intended to the details of construction or design herein shown, other than as described in the claims below. It is therefore evident that the particular illustrative embodiments disclosed above may be altered, combined, or modified and all such variations are considered within the scope of the present disclosure. The systems and methods illustratively disclosed herein may suitably be practiced in the absence of any element that is not specifically disclosed herein and/or any optional element disclosed herein. While compositions and methods are described in terms of “comprising,” “containing,” or “including” various components or steps, the compositions and methods can also “consist essentially of” or “consist of” the various components and steps. All numbers and ranges disclosed above may vary by some amount. Whenever a numerical range with a lower limit and an upper limit is disclosed, any number and any included range falling within the range is specifically disclosed. In particular, every range of values (of the form, “from about a to about b,” or, equivalently, “from approximately a to b,” or, equivalently, “from approximately a-b”) disclosed herein is to be understood to set forth every number and range encompassed within the broader range of values. Also, the terms in the claims have their plain, ordinary meaning unless otherwise explicitly and clearly defined by the patentee. Moreover, the indefinite articles “a” or “an,” as used in the claims, are defined herein to mean one or more than one of the elements that it introduces. If there is any conflict in the usages of a word or term in this specification and one or more patent or other documents that may be incorporated herein by reference, the definitions that are consistent with this specification should be adopted.

As used herein, the phrase “at least one of” preceding a series of items, with the terms “and” or “or” to separate any of the items, modifies the list as a whole, rather than each member of the list (i.e., each item). The phrase “at least one of” allows a meaning that includes at least one of any one of the items, and/or at least one of any combination of the items, and/or at least one of each of the items. By way of example, the phrases “at least one of A, B, and C” or “at least one of A, B, or C” each refer to only A, only B, or only C; any combination of A, B, and C; and/or at least one of each of A, B, and C.

The use of directional terms such as above, below, upper, lower, upward, downward, left, right, and the like are used in relation to the illustrative embodiments as they are depicted in the figures, the upward direction being toward the top of the corresponding figure and the downward direction being toward the bottom of the corresponding figure.

What is claimed is:

1. A surgical system, comprising:
a surgical instrument insertable through a first trocar into a body cavity of a patient, the surgical instrument comprising an end effector having opposing first and second jaws; and
a clip magazine insertable through a second trocar into the body cavity and including a surgical clip,
wherein the end effector is interactable with the clip magazine within the body cavity to load the surgical clip between the opposing first and second jaws.

2. The surgical system of claim 1, wherein the clip magazine comprises a body defining a slot sized to receive the surgical clip, and wherein the opposing jaws are receivable within the slot to load the surgical clip between the opposing first and second jaws.

3. The surgical system of claim 1, wherein the clip magazine comprises:
a shaft; and
a clip holder rotatably mounted to the shaft and defining a receptacle that receives the surgical clip, the clip holder being rotatable between a stowed position, where the clip holder is aligned with a longitudinal axis of the shaft, and a deployed position, where the clip holder is rotated out of alignment with the longitudinal axis.

4. The surgical system of claim 3, wherein the clip magazine further comprises an actuator movable relative to the shaft between a retracted position, where the clip holder is rotated to the stowed position, and an extended position, where the clip holder is rotated toward the deployed position.

5. The surgical system of claim 4, wherein:
the actuator comprises a rack comprising first teeth;
the clip holder comprises second teeth arranged to intermesh with the second teeth; and
the first teeth drive against the second teeth to rotate the clip holder between the stowed and deployed positions.

6. The surgical system of claim 5, wherein the surgical clip is a first surgical clip and the surgical system further includes a feeder system operable to load a second surgical clip into the clip holder after the first surgical clip is removed from the clip holder.

7. The surgical system of claim 6, wherein the second surgical clip is included in a plurality of serially arranged surgical clips, and the feeder system further includes:
a pusher plate engageable against the plurality of serially arranged surgical clips; and
a spring engageable against the pusher plate and operable to bias the pusher plate against the plurality of serially arranged surgical clips and thereby sequentially feed surgical clips of the plurality of serially arranged surgical clips into the receptacle of the clip holder.

8. The surgical clip of claim 7, wherein the clip holder is transitionable to the stowed state to receive a last one of the plurality of serially arranged surgical clips, and the deployed state, to allow the end effector to load the last one of the plurality of serially arranged surgical clips between the opposing first and second jaws.

9. A surgical system, comprising:
a surgical instrument insertable through a first trocar into a body cavity of a patient, the surgical instrument comprising and end effector having opposing first and second jaws; and
a clip magazine insertable through a second trocar into the body cavity and including:
a shaft extendable through the second trocar and into the body cavity; and
clip holder rotatably mounted to the shaft and providing a receptacle sized to hold a surgical clip,
wherein the end effector is interactable with the clip magazine within the body cavity to load the surgical clip between the opposing first and second jaws.

10. The surgical system of claim 9, wherein the clip holder is rotatable between a stowed position where the clip holder is aligned with a longitudinal axis of the shaft, and a deployed position, where the clip holder is rotated out of alignment with the longitudinal axis.

11. The surgical system of claim 10, wherein the clip magazine further comprises an actuator movable relative to the shaft between a retracted position, where the clip holder is rotated to the stowed position, and an extended position, where the clip holder is rotated toward the deployed position.

12. The surgical system of claim 11, wherein:
the actuator comprises a rack comprising first teeth;
the clip holder includes second teeth arranged to intermesh with the second teeth; and
the first teeth drive against the second teeth to rotate the clip holder between the stowed and deployed positions.

13. The surgical system of claim 9, wherein the surgical clip is a first surgical clip and wherein the surgical system further comprises a feeder system operable to load a second surgical clip into the clip holder after the first surgical clip is removed from the clip holder.

14. The surgical system of claim 9, wherein the clip holder is a first clip holder and the surgical clip is a first surgical clip, and wherein the clip magazine further comprises a second clip holder rotatably coupled to the shaft and including a receptacle sized to hold a second surgical clip.

15. The surgical system of claim 14, wherein the clip magazine further comprises an actuator movable relative to the shaft between a retracted position, where the first and second clip holders are each arranged in a stowed position, an extended position, where the first and second clip holders are rotated simultaneously toward a deployed position.

16. A method, comprising:
advancing an end effector of a surgical instrument into a body cavity of a patient via a first trocar, the end effector including opposing first and second jaws;
advancing a clip magazine into the body cavity of the patient via a second trocar, the clip magazine including a surgical clip;
moving at least one of the end effector and the clip magazine within the body cavity to place then end effector adjacent the clip magazine; and
loading the surgical clip between the first and second jaws within the body cavity.

17. The method of claim 16, wherein the clip magazine defines a slot sized to receive the surgical clip, and wherein loading the surgical clip between the first and second jaws comprises inserting the first and second jaws into the slot.

18. The method of claim 16, wherein the clip magazine comprises a clip holder that defines a receptable sized to receive the surgical clip, and wherein the method further comprises advancing an actuator through a shaft of the clip magazine, thereby rotating the clip holder toward a deployed state.

19. The method of claim 18, wherein loading the surgical clip between the first and second jaws comprises inserting the first and second jaws into the clip holder in the deployed state.

20. The method of claim 16, wherein the surgical clip comprises a first surgical clip and the clip magazine further includes a second surgical clip, the method further comprising:
applying the first surgical clip to tissue with the surgical instrument; and
loading the second surgical clip between the first and second jaws within the body cavity.
